(19) United States
(12) Patent Application Publication
(10) Pub. No.: US 2011/0270128 A1
(43) Pub. Date: Nov. 3, 2011

(54) RETRACTABLE BLOOD-SAMPLING DEVICE

(76) Inventors: Jilong Zhao, Zibo (CN); Qiang Wang, Zibo (CN)

(21) Appl. No.: 13/139,275

(22) PCT Filed: Dec. 8, 2009
(86) PCT No.: PCT/CN09/01386

§ 371 Date: Jun. 10, 2011

(30) Foreign Application Priority Data
Jan. 17, 2009 (CN) 200920018330.8

(51) Int. Cl. A61B 5/15

(52) U.S. Cl. 600/576

(57) ABSTRACT

A retractable blood-sampling device comprises a housing (15), a blood-sampling needle holder (13), a blood-sampling needle and a recovery sleeve (19). There is a compressible spring (14) mounted between the blood-sampling needle holder (13) and the front end of the housing (15). There is at least a hanging arm (16) on the blood-sampling needle holder (13), and the hanging arm (16) hangs on the front end of the housing (15). When the hanging arm (16) is subjected to an inward pressure, the hanging arm (16) disengages from the housing (15), the spring (14) is released, and the blood-sampling needle holder (13) and the blood-sampling needle are retraced into the recovery sleeve (19).

### BACKGROUND

1. Field of the Invention

This invention relates to a medical apparatus for drawing fluid, and especially to a retractable blood sampling device.

2. Description of Related Art

In clinical practice, double ended puncture needles and vacuum blood collection tubes are widely used in human blood sampling, and can be used for multiple times of blood collection on the same patient. The common housing barrel device for fixing the double ended puncture needle does not provide separation from or protection against the needle point exposed outside the blood sampling device after a blood sampling, and therefore the dangerously exposed needle may easily cause injuries to users, and may also cause injuries in disposal after use due to lack of effective protection.

Chinese Patent No. CN1368865A discloses a retractable blood sampling, which includes a housing barrel and a needle carrier inside said housing barrel which comprises several major parts. The needle carrier supports a double ended puncture needle. When said major parts of the housing barrel are rotated, the puncture needle may be retracted into the housing barrel by a spring and be contained in there to realize safe disposal. U.S. Pat. No. 5423758 discloses a device of similar structure, which uses frictional force fixed needle seating.

PCT application No. PCT/AU2005/000063 discloses a blood sampling device including: a housing barrel having an open end to accommodate a vacuum blood collection tube, a needle carrier at the closed end of housing barrel, a double ended puncture needle and a reclaim barrel. The needle carrier fixed on the housing barrel can be released so that it will be retracted back into the reclaim barrel after use. The reclaim barrel can be inserted into the housing barrel. The needle carrier has at least one hook that is placed into the housing barrel so that the needle carrier is fixed to the housing barrel. Hooks can move between its locking position and its release position. The reclaim barrel has an elongated vacuum chamber with one closed end and one open end sealed with a plunger. The plunger can cause the needle carrier slide in the vacuum chamber after the needle carrier being released.

### SUMMARY OF THE INVENTION

This invention provides a retractable blood sampling device, with a simple structure, and better compatibility, safety and reliability.

The detailed description is as follows:

A retractable blood sampling device comprising:

a housing barrel, with an open end and being able to accommodate a vacuum blood collection tube;

a needle holder, positioned on a front end of the housing barrel and with a needle seat placed thereon;

a needle, being fixed on the needle seat;

a recovery sleeve, a long cylinder with one open end and one sealed end and an outward protruding retainer ring at the open end, and used to accommodate the needle and the needle holder;

a compressible spring mounted between the needle holder and the front end of housing barrel,

wherein there is at least one hanging arm on the needle holder, said hanging arm hangs on the front end of the housing barrel, and when the hanging arm is subject to an inward pressure, the hanging arm disengages from housing barrel, the compressible spring is released, and the needle holder and the needle are retracted into the recovery sleeve.

Said housing barrel has one open end and one sealed end; wherein there is a surface at the sealed end, there is an opening in the center of the surface, and the opening slides with the needle holder; wherein on the side wall of the housing barrel front end there is a meshing structure engaging the hanging arm so that the needle holder is placed on the housing barrel; and wherein on the side wall of the housing barrel there is a projection for engaging the retainer ring, and said projection prevents the recovery sleeve from breaking off from the housing barrel when the recovery sleeve is injected to the bottom end of the housing barrel and the compressible spring is released.

The meshing structure is one or more projections on the side wall of the housing barrel front end, or a flange ring on the side wall of the housing barrel front end.

There is a window on the sealed surface between the center opening and a side wall of the housing barrel; and wherein said window is used to place a head of the hanging arm.

On said recovery sleeve there is flank to make sure the needle holder and recovery sleeve are coaxial so that needle and needle carrier remain steadfast in retracting.

Another installation of the hanging arm and the housing barrel for the present invention is: said housing barrel has one open end and one sealed end; wherein there is a surface at the sealed end, there is an opening at the center of the surface, and said opening slides with the needle holder; and wherein there is a window on the sealed surface between the center opening and the side wall, and the hanging arm passes through the window to hang on the housing barrel.

There is at least one hanging arm on the needle carrier, and said hanging arm is upwardly and outwardly inclined. When the hanging arm is vertical, its outer side has an inclined plane relative to the recovery sleeve so that the hanging arm is axially drawn in.

The needle seat has internal threads to match commonly used double ended puncture needles.

Another description of this invention is:

A retractable blood sampling device comprising: a housing barrel adapted to a vacuum blood collection tube, wherein an independent needle holder is placed on a front end of housing barrel, a needle seat is placed on the needle carrier, a hanging arm projected upwardly and outwardly is placed on the needle holder, with the top of the hanging arm hangs on the housing barrel, a compressible spring is placed between the needle holder and the housing barrel front end, and a touching barrel is provided corresponding to the hanging arm.

There is an opening on the housing barrel, there is a buckle hand on the top of the hanging arm, and the buckle hand hangs on the front end of the housing barrel.

The touching barrel is a one end sealed recovery sleeve with a retainer ring outside the open end, the front end of the housing barrel has a plate spring matching the retainer ring, and the top of the plate spring is inclined inwardly.

At the original position, the needle holder hangs on the housing barrel by the hanging arm. In use, a double ended needle is installed on the needle seat on the needle holder to take blood. After use, the touching barrel is used to move the hanging arm so that the hanging arm's head breaks off from the housing barrel and the needle holder breaks off from the housing barrel by the spring and the needle holder is collected to avoid exposure and remain safe.

There are many ways to connect the hanging arm's head to the housing barrel, for instance, a relatively easy way is to have an opening on the housing barrel and a buckle hand on the hanging arm used to hang on the housing barrel opening. Alternatively, a projection is placed on the housing barrel and a buckle hand hangs on the projection. Several hanging arms may be scattered circumferentially, e.g., 2-6 hanging beams.

The touching barrel can be a recovery sleeve, there is a retainer ring on its outside open end, and a plate spring is cut on the front end of housing barrel corresponding to the retainer ring. The top of the plate spring is inwardly inclined. While touching the hanging arm with an end sealed recovery sleeve, the needle holder breaks off into the recovery sleeve, the retainer ring is held by the housing barrel plate spring, and the end sealed recovery sleeve is connected to the housing barrel to confine the needle inside for the sake of safety.

Medical plastics are preferred for making the housing barrel, the needle holder and the recovery sleeve, by one time injection moulding as it is an easy method, and meanwhile medical plastics also meet requirement on elasticity.

Compared with the prior art, the advantages of this invention are:

1. Compared with the retracting force produced by vacuum, the retracting force provided by spring is stable in this invention.

2. Compared with the method of rotating a needle holder on the front end of housing barrel or using the force of friction to position a needle holder, the method of hanging needle holder by using a recovery sleeve to touch off a compressive spring is more reliable. Since the compressive spring and the needle holder are installed inside the housing barrel, they are effectively protected and less affected by outside factors.

3. The housing barrel gives effective protection to the spring device, providing high level of safety and reliability.

4. Compared with the prior art, this invention uses less parts, provides easier assembly, and is compatible to commonly used double ended needles.

### BRIEF DESCRIPTION OF THE DRAWING FIGURES

FIG. 1 is an illustrative diagram of a retractable blood sampling device according to a first embodiment of the present invention.

FIG. 2 is an illustrative diagram of a reclaim barrel in the retractable blood sampling device shown in FIG. 1.

FIG. 3 is a perspective view of the retractable blood sampling device shown in FIG. 1.

FIG. 4 is an illustrative diagram of a needle at its original state in the retractable blood sampling device shown in FIG. 1.

FIG. 5 is an illustrative diagram of the retractable blood sampling device shown in FIG. 1 when a spring touched off.

FIG. 6 is an illustrative diagram of a housing barrel according to a second embodiment of the present invention.

FIG. 7 is an illustrative diagram of a retractable blood sampling device according to a third embodiment of the present invention.

FIG. 8 is a perspective view of a needle carrier in the retractable blood sampling device shown in FIG. 7.

FIG. 9 is an illustrative diagram of a reclaim barrel according to a fourth embodiment of the present invention.

### DETAILED DESCRIPTION

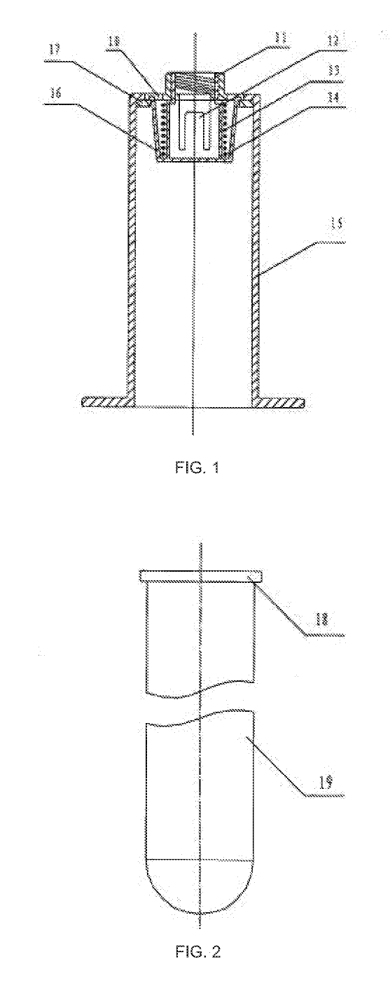

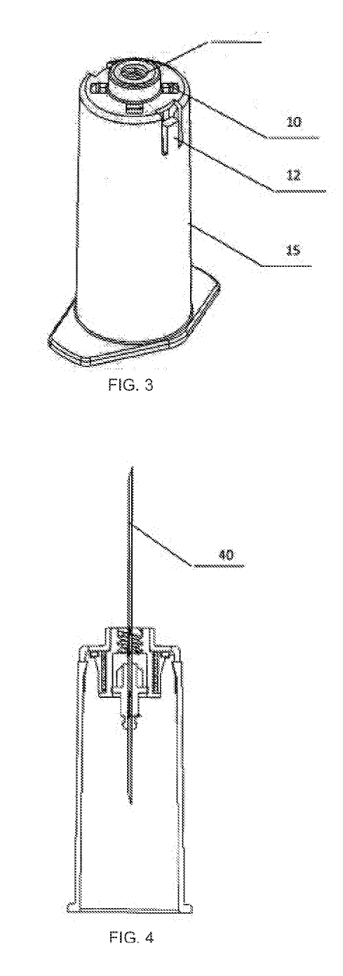

As shown in FIGS. 1, 2 and 3, a retractable blood sampling device has a housing barrel 15 adapted to a vacuum blood collection tube. On the front end of the housing barrel 15, there is a separated needle holder 13. There is a needle seat 11 on top of the needle holder 13. A hanging arm 16 projecting upward and outward is placed on the needle holder 13. and on the top end of housing barrel 15, there is a projection 17. On the front end of the hanging arm 16, there is a buckle hand hanging on the projection 17. Alternatively, an opening 10 may be made on the housing barrel 15 and the buckle hand may hang on the opening 10. Between the needle holder 13 and the front end of the housing barrel 15 is a compressive spring 14. A recovery sleeve 19 is provided for the hanging arm 16. Outside the open end of the recovery sleeve 19 is a retainer ring 18. A plate spring 12 is cut on the front end of housing barrel 15 to match the retainer ring 18 and the top of plate spring 12 is inwardly inclined.

One or more projections or flange rings on the internal wall of housing barrel 15 may be used to replace the plate spring 12. When recovery sleeve 19 is injected deep into the housing barrel 15, the compressive spring 14 is released, said projections or flange rings prevent the recovery sleeve 19 from breaking off the housing barrel 15.

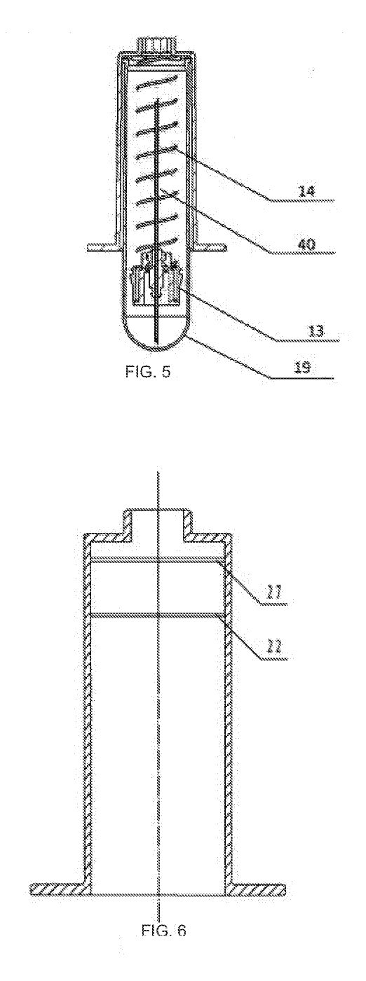

As shown in FIG. 4, in an original state, the needle holder 13 hangs on the housing barrel 15 by the hanging arm 16. In use, a double ended needle 40 is attached to needle seat 11 of the needle holder 13 to conduct blood collection. As shown in FIG. 5, after use, the hanging arm 16 may touch with the open end of the recovery sleeve 19 and the top of the hanging arm 16 withdraws to break off from the housing barrel 15, the needle holder 13 is broken off from the housing barrel 15 the compressive spring 14 and falls into the recovery sleeve 19. Meanwhile the retainer ring 18 is held fast by the housing barrel plate spring 12 so that the recovery sleeve 19 and the housing barrel 15 are connected together, sealing the double ended needle inside the end sealed recovery sleeve 19 for the sake of safety.

As shown in FIG. 6, the second embodiment of the present invention is similar to the first embodiment, except that on the internal wall of the front end of housing barrel 25 there are flange rings 27 and 22. The flange ring 27 is used to hang the needle holder while the flange ring 22 is used to prevent the recovery sleeve from breaking off from the housing barrel.

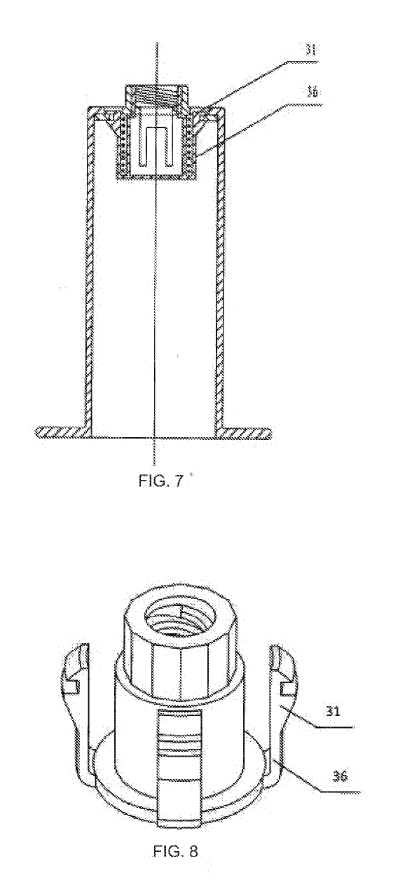

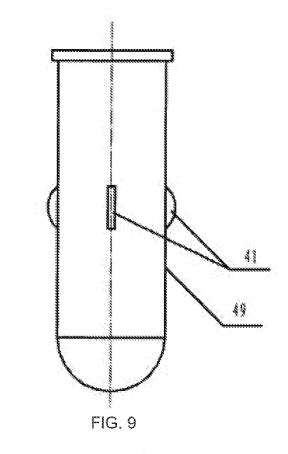

As shown in FIGS. 7 and 8, the third embodiment of the present invention is similar to the first and second embodiments, except that a hanging arm 36 is vertical and upward. There is an inclined plane 31 at the outside of the hanging arm 36, and the inclined plane 31 and a recovery sleeve make the hanging arm 36 to draw in axially.

As shown in FIG. 9, the fourth embodiment is similar to the first, second and third embodiments of the present invention, except that a recovery sleeve 49 has a flank 41 performing the function of axial positioning.

1. A retractable blood sampling device comprising:
a housing barrel, with an open end and being able to accommodate a vacuum blood collection tube;
a needle holder, positioned on a front end of the housing barrel and with a needle seat placed thereon;
a needle, being fixed on the needle seat;
a recovery sleeve, a long cylinder with one open end and one sealed end and an outward protruding retainer ring at the open end, and used to accommodate the needle and the needle holder; and
a compressible spring mounted between the needle holder and the front end of housing barrel,
wherein there is at least one hanging arm on the needle holder, said hanging arm hangs on the front end of the housing barrel, and when the hanging arm is subject to an inward pressure, the hanging arm disengages from housing barrel, the compressible spring is released, and the needle holder and the needle are retracted into the recovery sleeve.

2. The retractable blood sampling device of claim 1, wherein said housing barrel has one open end and one sealed end; wherein there is a surface at the sealed end, there is an opening in the center of the surface, and the opening slides with the needle holder; wherein on the side wall of the housing barrel front end there is a meshing structure engaging the hanging arm so that the needle holder is placed on the housing barrel; and wherein on the side wall of the housing barrel there is a projection for engaging the retainer ring, and said projection prevents the recovery sleeve from breaking off from the housing barrel when the recovery sleeve is injected to the bottom end of the housing barrel and the compressible spring is released.

3. The retractable blood sampling device of claim 2, wherein the meshing structure is one or more projections on the side wall of the housing barrel front end.

4. The retractable blood sampling device of claim 2, wherein the meshing structure is a flange ring on the side wall of the housing barrel front end.

5. The retractable blood sampling device of claim 3, wherein there is a window on the sealed surface between the center opening and a side wall of the housing barrel; and wherein said window is used to place a head of the hanging arm.

6. The retractable blood sampling device of claim 1, wherein said housing barrel has one open end and one sealed end; wherein there is a surface at the sealed end, there is an opening at the center of the surface, and said opening slides with the needle holder; and wherein there is a window on the sealed surface between the center opening and the side wall, and the hanging arm passes through the window to hang on the housing barrel.

7. The retractable blood sampling device of claim 1, wherein there is at least one hanging arm on the needle carrier, and said hanging arm is upwardly and outwardly inclined.

8. The retractable blood sampling device of claim 1, wherein there is at least one hanging arm on the needle holder, the outside of said hanging arm has an inclined plane, and the inclined plane and the recovery sleeve make the hanging arm to draw in axially.

9. The retractable blood sampling device of claim 1, wherein the needle seat has internal threads or a through hole.

10. The retractable blood sampling device of claim 1, wherein said recovery sleeve has a flank matching the internal surface of the housing barrel.

11. A retractable blood sampling device comprising: a housing barrel adapted to a vacuum blood collection tube, wherein an independent needle holder is placed on a front end of housing barrel, a needle seat is placed on the needle carrier, a hanging arm projected upwardly and outwardly is placed on the needle holder, with the top of the hanging arm hangs on the housing barrel, a compressible spring is placed between the needle holder and the housing barrel front end, and a touching barrel is provided corresponding to the hanging arm.

12. The retractable blood sampling device of claim 11, wherein there is an opening on the housing barrel, there is a buckle hand on the top of the hanging arm, and the buckle hand hangs on the front end of the housing barrel.

13. The retractable blood sampling device of claim 11, wherein the touching barrel is a one end sealed recovery sleeve with a retainer ring outside the open end, the front end of the housing barrel has a plate spring matching the retainer ring, and the top of the plate spring is inclined inwardly.

14. The retractable blood sampling device of claim 4, wherein there is a window on the sealed surface between the center opening and a side wall of the housing barrel; and wherein said window is used to place a head of the hanging arm.

15. The retractable blood sampling device of claim 7, wherein there is at least one hanging arm on the needle holder, the outside of said hanging arm has an inclined plane, and the inclined plane and the recovery sleeve make the hanging arm to draw in axially.

16. The retractable blood sampling device of claim 12, wherein the touching barrel is a one end sealed recovery sleeve with a retainer ring outside the open end, the front end of the housing barrel has a plate spring matching the retainer ring, and the top of the plate spring is inclined inwardly.
